(19) United States
(12) Patent Application Publication
(10) Pub. No.: US 2016/0220512 A1
(43) Pub. Date: Aug. 4, 2016

(54) MODIFIED RELEASE PHARMACEUTICAL FORMULATIONS

(71) Applicant: RUBICON RESEARCH PRIVATE LIMITED, Marg,Bhandup (West), Mumbai (IN)

(72) Inventors: Pratibha Sudhir PILGAONKAR, Mumbai (IN); Maharukh Tehmasp RUSTOMJEE, Mumbai (IN); Anilkumar Surendrakumar GANDHI, Mumbai (IN)

(21) Appl. No.: 15/021,384

(22) PCT Filed: Sep. 12, 2014
(86) PCT No.: PCT/IN2014/000592

§ 371 Date: Mar. 11, 2016

(30) Foreign Application Priority Data
Sep. 15, 2013 (IN) 802/MUM/2013

(51) Int. Cl. A61K 31/13; A61K 9/10; A61K 31/445; A61K 47/48; A61K 9/50

(52) U.S. Cl. CPC A61K 31/13; A61K 47/48184; A61K 9/5047; A61K 31/445; A61K 9/10

(57) ABSTRACT

The present invention relates to modified release compositions comprising plurality of modified release units. Particularly, the modified release units comprise at least one anti-Alzheimer's agent, at least one ion exchange resin and at least one release modifier.

### FIELD OF THE INVENTION

The present invention relates to modified release pharmaceutical formulations of Anti-Alzheimer's agents comprising plurality of modified release units comprising at least one Anti-Alzheimer's agent, at least one ion exchange resin, and at least one release modifier. Particularly, the modified release units comprise coated drug-resin complexes comprising drug-resin complexes of at least one active agent and at least one ion exchange resin, coated with at least one, release modifying agent. Further, the present invention also relates to a process for the preparation of the modified release pharmaceutical formulations.

### BACKGROUND OF THE INVENTION

Modified release dosage forms are designed to release a drug at a predetermined rate in body and are especially designed to reduce the frequency of drug administration, improve patient compliance, minimize side effects, and maintain desired drug levels for specific period of time. Modified release formulations can reduce peak trough plasma level fluctuations and improve efficacy of the drugs as compared to the immediate release dosage forms.

Alzheimer's disease (AD) is a progressive neurological disease of the brain characterized by irreversible loss of, neurons and the loss of intellectual abilities, including memory and reasoning that become severe enough to impede social or occupational functioning. Alzheimer's disease is the most common form of dementia. Clinically, Alzheimer's disease (AD) is characterized by beta-amyloid plaque depositions, tau pathology, inflammation, cerebrovascular damage, and cell death of cholinergic neurons. The lack of cortical acetylcholine results in deficient cholinergic functions in cortex and hippocampus causing cognitive impairment. The average duration of the disease is 10 years, during which afflicted persons progress from mild memory loss to the need for 24-hour supervision to total dependency and death. Although there is no cure for Alzheimer's disease, anti-Alzheimer's drugs are designed to treat and manage its symptoms. Currently the anti-Alzheimer's agents commonly used for management of Alzheimer's include memantine, donepezil, rivastigmine, galantamine, and tacrine.

Memantine or 1-amino-3, 5-dimethyladamantane is an analogue of 1-amino-cyclohexane which is a systemically active uncompetitive N-methyl d-aspartate (NMDA) receptor antagonist having moderate affinity for the receptor, strong voltage dependency and rapid blocking/unblocking kinetics. Memantine and other 1-amino alkyl hexanes have proven useful in treatment of moderate to severe dementia of the Alzheimer's disease. Memantine is also found to be effective for the treatment of autism, attention deficit/hyperactivity disorder (ADHD) and other autistic spectrum disorders, alleviation of various progressive neurodegenerative disorders such as dementia of Parkinson's disease, and spasticity. Memantine and its salts are highly soluble, highly permeable and are classified as BCS Class I compounds. After oral administration, the absolute bioavailability of memantine is 100%.

Dosing regimen of memantine as immediate release formulations for twice a day administration due to its high bioavailability can result in sudden dose dumping and cause considerable fluctuations in peak and trough plasma concentrations of the drug. Such rapid increase in blood plasma concentration levels immediately after administration of immediate release formulations of the drug can lead to undesirable side effects in patients. Compared to immediate release formulations, a modified release formulation containing a physiologically active drug allows blood concentrations of the drug to be maintained for a long time, thereby reducing the number of administrations and potentially improving patient compliance. Modified or sustained release compositions also lead to a decrease in the occurrence of concentration-related adverse effects and better tolerability as compared to immediate release formulations.

Furthermore for the people suffering from CNS disorders, particularly Alzheimer's disease a regimen with immediate release formulations is not optimal because the frequency of dosing may not only decrease patient compliance but in some cases the patients may also forget to take prescribed doses through the day. Therefore, again modified formulations that do not require multiple administrations and release the drug at a predetermined rate to maintain a desired therapeutic effect over a comparatively longer period of time with reduced side effects thereby increasing patient compliance, minimizing peak drug concentrations fluctuations in blood and improving effectiveness of the medication are particularly desirable. Further, it is also desirable that the bitter taste of memantine is masked in the formulation for better patient acceptance. However, design of modified release formulations of water soluble drugs such as mematine hydrochloride is a challenging task.

Some attempts have been made to provide modified release formulations of anti-Alzheimer's agents such as memantine hydrochloride wherein either a polymer coating is employed on the oral dosage form for providing modified release characteristics or a matrix type extended release approach is employed. PCT Publication WO2006/0009769 discloses modified release formulations comprising memantine hydrochloride and a polymeric “carrier” in the form of a coating and/or matrix. The formulations release at least 70-80% memantine in 4 to 24 hours in a “use environment” such as gastric fluid. The polymeric carrier when used as a polymeric matrix can be a hydrophilic or hydrophobic polymer. U.S. Pat. No. 5,382,601 provides solid pharmaceutical dosage forms containing memantine, which exhibit an extended two-phase release profile, with a portion of the drug being released immediately, followed by a sustained release of the remainder. The matrix of this formulation contains both a water-soluble and a water-insoluble salt of casein preferably sodium and calcium caseinate. However, casein tends to display instability in varying pH. PCT Publication WO2010/112221 discloses pharmaceutical compositions comprising a non-swelling polymer matrix component having dispersed therein memantine hydrochloride and a pore-forming agent. The non-swelling polymer matrix component comprises a polyvinylacetate polymer and the pore-forming agent is polyvinyl pyrrolidone. U.S. Patent Publication 2010/0272794 discloses a composition comprising memantine, a lipidic drug release rate controlling system and excipients providing extended release of the drug. The lipidic drug release rate controlling substance is used as a core forming substance which is structured as a matrix.

However with the use of film forming polymers to form a release retarding coat on the oral dosage form, there exist chances of premature rupture of the coat either by chewing, splitting or abrasion, resulting in release of an excessive amount of active ingredient, leading to undesirable effects. With matrix type delivery systems, a complete release of drug from the matrix tablet may not be achieved in practice. Therefore need exists for modified release compositions of multi-particulate type for memantine hydrochloride that overcomes the above drawbacks, releases the active over an extended period of time and maintains therapeutically effective plasma levels.

PCT Publication WO2006/138227 discloses pharmaceutical beads of memantine having immediate or modified release properties. The beads may be filled into a capsule or compressed into a tablet. Exemplified modified release beads comprise an inert core, a drug containing layer with binder and glidant, optionally a seal coat layer, a modified release layer, and optionally a topcoat. PCT Publication WO2012/110912 discloses a sustained release composition comprising a core containing memantine which is coated with a water insoluble substance and a water soluble substance in a specific ratio. PCT Publication WO2012/101653 discloses composition, which exhibits a biphasic release profile; comprising modified release component and at least one immediate release component and more than 3% by weight of binders. The modified release comprises a plurality of sustained release components comprising memantine and rate controlling polymers. The immediate release component comprising memantine is coated over the sustained release components. U.S. Publication 2007/0065512 discloses a modified and immediate release pharmaceutical dosage forms containing memantine that exhibit a predetermined enhanced release profile and absorption. The composition disclosed comprises a plurality of beads, each bead comprising an inert core; and a mixture of memantine as and a polymer binder coated on said inert core.

Therefore though some modified release compositions of memantine have been suggested, there still remains a need to have an alternate multi-particulate modified or extended release composition of memantine hydrochloride. Such compositions should also be simple and economical to manufacture and reproducible. Particularly there exists a need to provide formulations wherein the in-vitro release profile of memantine can be modulated as desired to not cause any sudden excessive increases in the peak plasma levels while also preventing incomplete release of the active from the dosage form. Need also exists to provide modified release formulations of memantine hydrochloride in the form of palatable solid or liquid formulations to improve patient compliance and convenience.

The present inventors after excessive efforts and experimentation have developed modified release pharmaceutical formulations comprising plurality of modified release units wherein each unit comprises drug-resin complexes comprising complexes of at least one active agent and at least one ion-exchange resin, coated with at least one release modifying coating agent. The modified release compositions of the present invention provide release of the active agent at a predetermined rate thereby avoiding any undesired rapid release of the drug which can lead to undesirable side effects while also avoiding incomplete or substantial release of the drug from the modified release pharmaceutical formulation. Moreover the formulations of the present invention can be presented in palatable solid or liquid formulation wherein the bitterness of the active agent is also masked. The modified release formulations of the present invention can also be provided in formulations such as liquid suspensions or rapidly disintegrating dosage forms and the like whereby the patients suffering from Parkinsons or Alzheimer's disease having trouble in swallowing tablets or capsules can also benefit from the formulations of the present invention.

### SUMMARY OF THE INVENTION

The present invention relates to modified release compositions comprising plurality of modified release units comprising at least one Anti-Alzheimer's agent, at least one ion exchange resin, and at least one release modifier. Particularly, the modified release units comprise coated drug-resin complexes comprising (a) drug-resin complex comprising at least one active agent and at least one ion-exchange resin; and (b) modified release coating.

### DETAILED DESCRIPTION OF THE INVENTION

The present invention relates to modified release pharmaceutical formulations comprising plurality of modified release units comprising at least one Anti-Alzheimer's agent, at least one ion exchange resin, and at least one release modifier. Particularly each modified release unit comprises drug-resin complexes comprising complexes of at least one Anti-Alzheimer's agent and at least one ion-exchange resin, coated with at least one release modifying coating agent. In one embodiment, the modified release formulation of the present invention comprise plurality of modified release units comprising coated drug-resin complexes comprising: (a) drug-resin complex comprising at least one Anti-Alzheimer's agent and at least one ion-exchange resin; and (b) modified release coating.

### Active Agents

The term “active agent/s” as employed herein refers to any suitable drug which can form a complex with ion exchange resins, and for which sustained release is desired. In general, all the drugs including, but not limited to, acidic, basic, amphoteric or zwitterionic drugs, especially those having high absolute bioavailability, fast rate of absorption, and high dissolution and permeability are desirable active agents for the invention. Particularly, the active drugs employed herein are anti-Alzheimer's agent or anti-Parkinson's drug.

The term “anti-Alzheimer's agent” or “anti-Alzheimer agent”, as employed herein refers to any compound that can be employed for the treatment of Alzheimer's disease and other dementias; such as, but not limited to, N-methyl-D-aspartate receptor (NMDA) receptor antagonists, acetyl cholinesterase inhibitor, acetylcholine synthesis modulators, acetylcholine storage modulators, acetylcholine release modulators, Aβ inhibitors, Aβ plaque removal agents, inhibitors of Aβ plaque formation, inhibitors of amyloid precursor protein processing enzymes, β-amyloid converting enzyme inhibitors, β-secretase inhibitors, γ-secretase modulators, nerve growth factor agonists, hormone receptor blockade agents, neurotransmission modulators, and combinations thereof. In one embodiment, the anti-Alzheimer's agent is an NMDA receptor antagonist. In one embodiment, the NMDA receptor antagonist includes, but not limited to, memantine, amantadine, neramexane (1, 3, 3, 5, 5-pentamethylcyclohexan-1-amine), ketamine, rimantidine, eliprodil, ifenprodil, dizocilpine, remacemide, riluzole, aptiganel, phencyclidine, flupirtine, celfotel, felbamate, spermine, spermidine, levemopamil, and/or combinations thereof. In another embodiment, NMDA receptor antagonist employed in the present invention is an Anti-Alzheimer agent. In one embodiment, the anti-Alzheimer's agent is an inhibitor of cholinesterase. In one embodiment, the acetylcholinesterase inhibitor includes, but is not limited to, donepezil, tacrine, rivastigmine, galantamine, physostigmine, neostigmine, Huperzine A, icopezil (CP-118954, 5,7-dihydro-3-[2-[1-(phenylmethyl)-4-piperidinyl]ethyl]-6H-pyrrolo-[4,5-f]-1,2-benzisoxazol-6-one maleate), ER-127528 (4-[(5,6-dimethoxy-2-fluoro-1-indanon)-2-yl]methyl-1-(3-fluorobenzyl) piperidine hydrochloride), zanapezil (TAK-147; 3-[1-(phenylmethyl)piperidin-4-yl]-1-(2,3,4,5-tetrahydro-1H-1-benzazepin-8-yl)-1-propane fumarate), metrifonate (T-588; −) (—R-α-[[2-(dimethylamino)ethoxy]methyl]benzo[b]thiophene-5-methanol hydrochloride), FK-960 (N-(4-acetyl-1-piperazinyl)-p-fluorobenzamide-hydrate), TCH-346 (N-methyl-N-2-pyropinyldibenz[b,f]oxepine-10-methanamine), SDZ-220-581 ((S)-α-amino-5-(phosphonomethyl)-[1,1′-biphenyl]-3-propionic acid), and combinations thereof. In another embodiment, the anti-Alzheimer's agent is an Aβ inhibitor, Aβ plaque removal agents, inhibitors of Aβ plaque formation, inhibitors of amyloid precursor protein processing enzymes, β-amyloid converting enzyme inhibitors, β-secretase inhibitors, γ-secretase modulators. In another embodiment, the Aβ inhibitor includes, but is not limited to, tarenflurbil, tramiprosate, clioquinol, PBT-2 and other 8-hydroxyquinilone derivatives, Aβ plaque removal agents, inhibitors of Aβ plaque formation, inhibitors of amyloid precursor protein processing enzymes, β-amyloid converting enzyme inhibitors, β-secretase inhibitors, γ-secretase modulators (LY450139; N—[N-(3,5-difluorophenacetyl)-L-alanyl)-S-phenylglycine t-butyl ester), and combinations thereof. In another embodiment, the anti-Alzheimer's agent is a nerve growth factor agonist. In another embodiment, the nerve growth factor agonist is, but not limited to, xaliproden or brain derived neurotrophic factor or nerve growth factor. In another embodiment, the anti-Alzheimer's agent is a hormone receptor blockade agent. In another embodiment, the hormone receptor blockade agent is, but not limited to, leuproelide or a derivative thereof. In another embodiment, the anti-Alzheimer's agent is a neurotransmission modulator. In another embodiment, the neurotransmission modulator is, but not limited to, ispronicline. The anti-Alzheimer agent employed in the compositions of the present invention may be in the form of free base, free acid or pharmaceutically acceptable salts, esters, prodrugs, metabolites, active metabolites, polymorphs, solvates, hydrates, derivatives, enantiomers, optical isomers, tautomers or racemic mixtures thereof. Suitable salts include, but are not limited to, acid addition salts, such as those made with hydrochloric, hydrobromic, hydroiodic, methylsulfonic, perchloric, sulfuric, nitric, phosphoric, acetic, propionic, glycolic, lactic pyruvic, malonic, succinic, maleic, fumaric, maleic, tartaric, citric, benzoic, carbonic cinnamic, mandelic, methanesulfonic, ethanesulfonic, benezenesulfonic, hydroxyethanesulfonic, p-toluene sulfonic, cyclohexanesulfamic, salicyclic, p-aminosalicylic, 2-phenoxybenzoic, and 2-acetoxy benzoic acid. All such salts are acceptable provided that they are non-toxic and do not substantially interfere with the desired pharmacological activity. In one embodiment the anti-Alzheimer agent employed in the present invention is memantine in the form of free base, free acid, or its pharmaceutically acceptable salts, prodrugs, esters, polymorphs, solvates, hydrates, metabolites, active metabolites, derivatives, enantiomers, optical isomers, tautomers or racemic mixtures. In one embodiment, the anti-Alzheimer agent employed in the compositions of the present invention is an NMDA receptor antagonist memantine in the form of free base or its pharmaceutically acceptable salts, prodrugs, esters, polymorphs, solvates, hydrates, metabolites, active metabolites, derivatives, enantiomers, optical isomers, tautomers or racemic mixtures. In one embodiment, the anti-Alzheimer agent employed in the compositions of the present invention is memantine hydrochloride.

Pharmaceutically effective amount of active is employed in the composition of the present invention. The term “effective amount” refers to an amount effective to achieve desired preventive, therapeutic and/or beneficial effect. In one embodiment the amount of anti-Alzheimer agent in the composition can vary from about 0.001 weight % to about 95 weight %, based on the total weight of the composition. In another embodiment the amount of anti-Alzheimer agent in the composition can vary from about 0.01 weight % to about 90 weight %, based on the total weight of the composition. In still another embodiment, the amount of anti-Alzheimer agent in the composition can vary from about 0.02 weight % to about 85 weight %, based on the total weight of the composition. In one embodiment the compositions of the present invention may be administered at a dose of about 0.01 mg to about 300 mg of anti-Alzheimer agent. In another embodiment the compositions of the present invention may be administered at a dose of about 0.1 mg to about 250 mg of anti-Alzheimer agent. In still another embodiment the compositions of the present invention may be administered at a dose of about 0.5 mg to about 200 mg of anti-Alzheimer agent. In one embodiment the compositions of the present invention may be administered at a dose of about 1 mg to about 100 mg of memantine hydrochloride. In one embodiment the compositions of the present invention may be administered at a dose of about 2 mg to about 75 mg of memantine hydrochloride.

In one embodiment at least one anti-Alzheimer's agent present in the plurality of multiple units employed in the compositions of the present invention is present in the form of a complex with at least one ion exchange resin. In another embodiment at least one anti-Alzheimer's agent may be present within or outside the plurality of multiple units in a non-complexed form.

### Ion Exchange Resins

The multiple units employed in the compositions of the present invention comprise at least one ion exchange resin. In one embodiment, the active agent employed in the compositions of the present invention is complexed with at least one ion exchange resin. In another embodiment, the ion exchange resin is present in the composition of the present invention in a non-complexed form. Ion-exchange resins are water-insoluble, cross-linked polymers containing covalently bound salt forming groups in repeating positions on the polymer chain. The ion-exchange resins suitable for compositions of the present invention consist of a pharmacologically inert organic and/or inorganic matrix. The matrixes contain functional groups that are ionic or capable of being ionized under the appropriate conditions of pH. The organic matrix may be synthetic such as, but not limited to, polymers or copolymers of acrylic acid, methacrylic acid, sulfonated styrene, sulfonated divinylbenzene, or partially synthetic such as, but not limited to, modified cellulose and dextrans. The matrix can also be inorganic, such as, but not limited to, silica gel, or aluminosilicates, natively charged or modified by the addition of ionic groups. The covalently bound ionic groups may be strongly acidic (e.g., sulfonic or sulfate acid groups), weakly acidic (e.g., carboxylic acid), strongly basic (e.g., quaternary ammonium), weakly basic (e.g., primary amine), or a combination of acidic and basic groups. Other types of charged groups can also be used, including any organic group that bears an acidic or a basic functional group, for example, an amine, imine, imidazoyl, guanidine, pyridinyl, quaternary ammonium, or other basic group, or a carboxylic, phosphoric, phenolic, sulfuric, sulfonic or other acidic group. The ion exchange resin having the polymeric matrix with an anionic functional group is a cation exchange resin and that having a cationic functional group is an anionic exchange resin. The mobile or exchangeable moieties depending on the type of resin can be but not limiting to sodium, hydrogen, potassium, chloride and the like.

In one embodiment of the present invention ion exchange resin employed is a cation exchange resin or an anion exchange resin or combination thereof. In a further embodiment, cation exchange resin is employed for complexation with the active agent. Non limiting examples of suitable cation exchange resin that may be employed include a copolymer of methacrylic acid and divinylbenzene, sodium polystyrene sulfonate resin, sulfonated copolymer of styrene and divinylbenzene, crosslinked polyacrylic acid resin, polyacrylate resin, crosslinked carboxylic acid resin, crosslinked sulfonic acid resin, crosslinked phosphonic acid resin, zeolite or a combination thereof.

In another embodiment, cation exchange resin that may be employed include, but are not limited to, Amberlite® IRP64 (porous copolymer of methacrylic acid and divinylbenzene), Amberlite® IRP69 (sodium polystyrene sulfonate or sulfonated copolymer of styrene and divinylbenzene), Amberlite® IRP88 (cross linked polymer of methacrylic acid and divinylbenzene), DOWEX® resins (strong cationic exchangers based upon polystyrenesulphonic acid with variable crosslinking (1-12% divinylbenzene)), Tulsion® 335-(Polacrilex/{Polacirilex S), Tulsion® 339 (Polacrilin potassium USP), Tulsion® 344 (Sodium polystyrene sulfonate BP), Indion® 204 (crosslinked polyacrylic acid), Indion® 214 (crosslinked polyacrylic acid), Indion® 234 (crosslinked polyacrylic acid), Indion® 234S (crosslinked polyacrylic acid), Indian® 294 (crosslinked polyacrylic acid), Purolite® C115 HMR (carboxylic acid functional group), Purolite® C115 E (carboxylic acid functional group), Purolite® C100 HMR (sulfonic acid functional group), Purolite® 100 MR (sulfonic acid functional group), polyacrylate resins, cation exchange resins having phosphonic functional groups or zeolites. Cationic exchange resins are selected for use with basic active agents and molecules having a cationic functionality. Other suitable ion-exchange resins include anion exchange resins, such as have been described in the art and are commercially available.

The size of the ion-exchange particles that may be employed in the compositions of the present invention may be from about 5 microns to about 750 microns. In one aspect the particle size is within the range of about 40 microns to about 250 microns for liquid dosage forms although particles up to about 1,000 micron can be used for solid dosage forms, e.g., tablets and capsules. Both regularly and irregularly shaped resin particles may be employed in the present invention. Regularly shaped particles are those particles that substantially conform to geometric shapes such as spherical, elliptical, and cylindrical and the like, which are exemplified by Dow XYS-40010.00 and Dow XYS-40013.00 (The Dow Chemical Company). Irregularly shaped particles are all particles not considered to be regularly shaped, such as particles with amorphous shapes and particles with increased surface areas due to surface channels or distortions. Irregularly shaped ion-exchange resins of this type are exemplified by Amberlite IRP-69 (Rohm and Haas).

In one embodiment, the ion exchange resin used in the compositions of the present invention is sodium polystyrene sulfonate.

### Drug-Resin Complexes

Drug-resin complexes according to the present invention comprise at least one active agent and at least one ion-exchange resin. In one embodiment basic active agent is complexed with cation exchange resin. In one embodiment anti-Alzheimer agent is complexed with ion-exchange resin. In another embodiment basic anti-Alzheimer agent is complexed with cation exchange resin. In still another embodiment memantine hydrochloride is complexed with a cation exchange resin. In another embodiment, anti-Alzheimer agent is complexed with sodium polystyrene sulfonate. In another embodiment memantine hydrochloride is complexed with sodium polystyrene sulfonate. In a still another embodiment active agent can be complexed with ion exchange resin in any weight or molar ratio. In a further embodiment, ion exchange resin can be used for complexation with active agent in a ratio of active agent to resin of about 1:0.1 to about 1:20. In another embodiment, ion exchange resin can be used for complexation with active agent in a ratio of active agent to resin of about 1:0.25 to about 1:10. In still another embodiment, ion exchange resin can be used for complexation with active agent in a ratio of active agent to resin of about 1:0.5 to about 1:5. Without being bound to any theory it is believed that the ion exchange resin employed in the present invention are for modifying the release of the active, masking any bitter taste of the active and also preventing the leaching of the active through the coat into the environment of use or of the formulation.

The drug-ion exchange resin complexes or drug-resin complexes can be prepared by methods known in the art, such as, but not limiting to, blending, slurrying, kneading, grinding, sieving, filling, compressing, lyophilization, spray-drying, fluid-bed drying or centrifugal granulation. The drug-resin binding may be performed, for example, as a batch or column process, as is known in the art. In one illustrative embodiment, drug-resin complex is prepared by batch process. In one embodiment the drug-resin complexes were prepared by stirring aqueous slurry of drug and ion exchange resin for about 0.5 hours to about 12 hours, followed by filtration and drying of the formed drug-resin complex.

In one embodiment, the invention relates to compositions comprising drug-resin complexes having one or more active agents. In another embodiment, the invention also relates to pharmaceutical compositions comprising drug-resin complexes wherein at least one pharmaceutically acceptable excipient has been employed during the process of preparation of the drug-resin complexes, such as, but not limited to, stabilizers and the like to inhibit or prevent degradation of the drug-resin complex during manufacturing process and over shelf life of the composition. Suitable stabilizers include, but are not limited to, antioxidants, chelating agents or combinations thereof. In one embodiment, stabilizer employed during the process of preparation of the drug-resin complexes is an antioxidant. Any suitable antioxidant agent available to those of ordinary skill in the art may be used. Antioxidant such as, but, not limited to ascorbic acid, sodium metabisulphite, potassium metabisulfite, sodium bisulfite, sodium sulfite, tocopherol, sorbic acid, retinol, butylated hydroxyanisole (BHA), butylated hydroxytoluene (BHT), propyl gallate, sodium benzoate or any salt thereof, or a combination thereof may be employed. Any suitable chelating agent known to those of ordinary skill in the art may be used. Chelating agents such as, but not limited to, ethylene diaminetetraacetic acid (EDTA), desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid, bis(aminoethyl)glycolether-N,N,N′,N′-tetraacetic acid, iminodiacetic acid, citric acid, tartaric acid, fumaric acid, or any salt thereof, or a combination thereof may be employed. The stabilizing agent can be present in a concentration of about 0.001% to 20% by weight of the composition and may or may not be in the final composition.

Further, the drug-resin complexes are impregnated with a solvating agent. The solvating agent can be added as an ingredient in the resin drug complexation step or the drug-resin complexes can be treated with the solvating agent after complexing. This treatment helps particles retain their geometry, and enables the effective application of barrier coatings to the drug-resin complexes resulting in the ability to effectively prolong the release of drugs from drug resin complexes. Solvating agent that can be employed in the compositions of the present invention include, but are not limited to, polyethylene glycol, propylene glycol, mannitol, lactose, methylcellulose, hydroxypropylmethylcellulose, sorbitol, polyvinylpyrrolidone, carboxypolymethylene, xanthan gum, propylene glycol alginate and combinations thereof. In one embodiment the solvating agent is polyethylene glycol. In one embodiment, the drug-resin complexes may not be impregnated with a solvating agent. In a further embodiment, the formulation may comprise at least two populations of solvated and non-solvated drug-resin complexes. In one embodiment, the formulation may comprise at least two populations of solvated and non-solvated drug resin complexes in a ratio from about 1:99 to about 99:1.

In an embodiment of the present invention along with the solvating agent, at least one or more pharmaceutically acceptable excipients, such as but not limited to stabilizers may be employed for impregnation of the drug-resin complexes. The stabilizers that may be employed during impregnation of the drug-resin complexes include the ones as described above under drug-resin complexes.

### Release Modifying Coating

The multiple units present in the compositions of the present invention comprise at least one anti-Alzheimer's agent, at least one ion exchange resin and at least one release modifier or release modifying agent. In one embodiment, the release modifier or release modifying agent is present in the multiple units of the present invention in the form of coating. In one embodiment the drug-ion exchange resin complexes or impregnated drug-resin complexes are coated with release modifying coating. In one embodiment, the entire portion of the drug-ion exchange resin complex or impregnated drug-resin complex is coated with the release modifying coating. In another embodiment, only a part of the drug-ion exchange resin complex or impregnated drug-resin complex is coated with the drug-ion exchange resin complex. The release modifying coating comprises at least one release modifying coating agent. The release modifier or release modifying coating agent can provide controlled release of active agent. The drug-resin complexes or impregnated drug-resin complexes are coated with a diffusion barrier coating of at least one release modifying agent coating. The modified release coating agent that may be employed for modified release coating in the compositions of the present invention includes, but are not limited to, water-insoluble or water-soluble, release modifiers or release modifying agents or combinations thereof. The modified release coating agent that may be employed for modified release coating in the compositions of the present invention includes, but are not limited to, polymeric and non-polymeric release modifiers or release modifying agents or combinations thereof.

The water-insoluble modified release coating agents that may be employed include polymeric water-insoluble modified release coating agents or non-polymeric water-insoluble modified release coating agents or combinations thereof. Suitable polymeric water-insoluble modified release coating agents include, but are not limited to, polyvinyl acetate, polyvinyl chloride, polyvinyl carbonate, ethyl cellulose, nitrocellulose, vinylidene chloride-acrylonitrile copolymer, acrylonitrile-styrene copolymer, ethylene vinyl acetate, cellulose acetate, cellulose acetate phthalate, cellulose acetate butyrate, copolymers of vinyl pyrrolidone, blend of polymers comprising polyvinyl acetate, hydroxypropylmethylcellulose phthalate, methacrylic acid copolymers such as Eudragit® LI00/SI00/LI00-55 and the like or mixtures thereof; methacrylate copolymers such as Eudragit® E100/EPO, Eudragit® RL100/RL30D/RLPO, Eudragit® RS100/RS30D/RSPO and the like or mixtures thereof. Suitable non-polymeric water-insoluble modified release coating agents include, but are not limited to, fats, oils, waxes, fatty acids, fatty acid esters, glycerides, long chain monohydric alcohols and their esters, phospholipids, terpenes or combinations thereof. The non-polymeric water-insoluble modified release coating agents employed in the compostions of the present invention include, but are not limited to, Cutina®) (hydrogenated castor oil), Hydrobase® (hydrogenated soybean oil), Castorwax® (hydrogenated castor-oil), Croduret® (hydrogenated castor oil), Carbowax® Compritol® (glyceryl behenate), Sterotex® (hydrogenated cottonseed oil), Lubritab® (hydrogenated cottonseed oil), Apifil® (wax yellow), Akofine® (hydrogenated cottonseed oil), Softisan® (hydrogenated palm oil), Hydrocote® (hydrogenated soybean oil), Corona® (Lanolin), Gelucire® (macrogolglycerides Lauriques), Precirol® (glyceryl palmitostearate), Emulcire™ (cetyl alcohol), Plurol® diisostearique (polyglyceryl diisostearate), Geleol® (glyceryl stearate), and mixtures thereof. In another embodiment, lipids or waxes can also be employed in the form of an aqueous dispersion stabilized by surfactants and suitable stabilizers. Suitable water soluble modified release coating agents that may be employed include, but are not limited to, polyvinylpyrrolidone, poloxamer, guar gum, xanthan gum, gum arabic, tragacanthan, cellulose derivatives such as hydroxypropylmethylcellulose, hydroxypropyl cellulose, methylcellulose, and hydroxyethyl cellulose, carboxymethylethyl cellulose, hydroxyethylmethyl carboxymethyl cellulose, hydroxyethyl methyl cellulose, carboxymethyl cellulose, methylhydroxyethyl cellulose, methylhydroxypropyl cellulose or any mixtures thereof.

Suitable polymeric release modifiers may be employed in the compositions of the present invention. According to the present invention, polymeric release modifier may be pH independent or pH dependent or any combination thereof. Polymeric release modifiers that are pH dependent exhibit pH dependent solubility, and hence their performance depends on the pH of the environment they encounter. Polymeric release modifiers that are pH independent exhibit solubility that is independent of pH and hence its performance does not depend on the pH of the environment they encounter. The polymeric release modifier employed in the compositions of the present invention may be swelling or non-swelling or any combinations thereof. The polymeric release modifier employed in the compositions of the present invention may be water soluble or water insoluble or any combinations thereof. In one embodiment, polymeric release modifiers that may be employed in the compositions of the present invention include, but are not limited to cellulose derivatives, saccharides or polysaccharides, poly(oxyethylene)-poly(oxypropylene) block copolymers (poloxamers), vinyl derivatives or polymers or copolymers thereof, polyalkylene oxides and derivatives thereof, maleic copolymers, acrylic acid derivatives or the like or any combinations thereof. Cellulose derivatives include, but are not limited to, ethyl cellulose, methylcellulose, hydroxypropylmethylcellulose (HPMC), hydroxypropyl cellulose (HPC), hydroxyethyl cellulose, hydroxymethyl cellulose, hydroxypropyl ethylcellulose, carboxymethylethyl cellulose, carboxy ethylcellulose, carboxymethyl hydroxyethylcellulose, hydroxyethylmethyl carboxymethyl cellulose, hydroxyethyl methyl cellulose, carboxymethyl cellulose, methylhydroxyethyl cellulose, methylhydroxypropyl cellulose, carboxymethyl sulfoethyl cellulose, sodium carboxymethyl cellulose, cellulose acetate, cellulose acetate phthalate, cellulose acetate butyrate, hydroxypropylmethylcellulose acetate succinate, hydroxypropylmethylcellulose phthalate, hydroxymethyl ethylcellulose phthalate, cellulose acetate phthalate, cellulose acetate succinate, cellulose acetate maleate, cellulose acetate trimelliate, cellulose benzoate phthalate, cellulose propionate phthalate, methylcellulose phthalate, ethylhydroxy ethylcellulose phthalate, or combinations thereof. Saccharides or polysaccharides include, but are not limited to, guar gum, xanthan gum, gum arabic, tragacanth or combinations thereof. Vinyl derivatives, polymers and copolymers thereof include, but are not limited to, polyvinylacetate aqueous dispersion (Kollicoat® SR 30D), copolymers of vinyl pyrrolidone, copolymers of polyvinyl alcohol, mixture of polyvinyl acetate and polyvinylpyrrolidone (e.g. Kollidon® SR), polyvinyl alcohol phthalate, polyvinylacetal phthalate, polyvinyl butylate phthalate, polyvinylacetoacetal phthalate, polyvinylpyrrolidone (PVP), or combinations thereof. Polyalkylene oxides and derivatives thereof include, but are not limited to, polyethylene oxide and the like or any combinations thereof. Acrylic acid derivatives include, but are not limited to, methacrylic acids, polymethacrylic acids, polyacrylates, especially polymethacrylates like a) copolymer formed from monomers selected from methacrylic acid, methacrylic acid esters, acrylic acid and acrylic acid esters b) copolymer formed from monomers selected from butyl methacrylate, (2-dimethylaminoethyl)methacrylate and methyl methacrylate c) copolymer formed from monomers selected from ethyl acrylate, methyl methacrylate and trimethylammonioethyl methacrylate chloride or d) copolymers of acrylate and methacrylates with/without quarternary ammonium group in combination with sodium carboxymethylcellulose, e.g. those available from Röhm GmbH under the trademark Eudragit® like Eudragit® EPO (dimethylaminoethyl methacrylate copolymer; basic butylated methacrylate copolymer), Eudragit® RL and RS (trimethylammonioethyl methacrylate copolymer), Eudragit® NE30D and Eudragit® NE40D (ethylacrylate methylmethacrylate copolymer), Eudragit® L 100 and Eudragit® S (methacrylic acid.methyl methacrylate copolymer), Eudragit® L 100-55 (methacrylic acid.ethyl acrylate copolymer), Eudragit® RD 100 (ammoniomethacrylate copolymer with sodium carboxymethylcellulose); or the like or any combinations thereof. Maleic copolymer based polymeric release modifier includes, but is not limited to, vinylacetate.maleic acid anhydride copolymer, styrene.maleic acid anhydride copolymer, styrene.maleic acid monoester copolymer, vinylmethylether.maleic acid anhydride copolymer, ethylene.maleic acid anhydride copolymer, vinylbutylether.maleic acid anhydride copolymer, acrylonitrile.methyl acrylate.maleic acid anhydride copolymer, butyl acrylate.styrene.maleic acid anhydride copolymer and the like, or combinations thereof.

The non-polymeric release modifier employed in the compositions of the present invention refers to any excipient that can retard the release of an active agent and that does not comprise of repeating units of monomers. Suitable non-polymeric release modifiers employed in the present invention include, but are not limited to, fatty acids, long chain alcohols, fats and oils, waxes, phospholipids, eicosonoids, terpenes, steroids, resins and the like or combinations thereof. Non-polymeric release modifiers employed may be pH dependent or pH independent in nature.

Fatty acids are carboxylic acids derived from or contained in an animal or vegetable fat or oil. Fatty acids are composed of a chain of alkyl groups containing from 4 to 22 carbon atoms and are characterized by a terminal carboxyl group. Fatty acids useful in the present invention include, but are not limited to, hydrogenated palm kernel oil, hydrogenated peanut oil, hydrogenated palm oil, hydrogenated rapeseed oil, hydrogenated rice bran oil, hydrogenated soybean oil, hydrogenated sunflower oil, hydrogenated castor oil, hydrogenated cottonseed oil, and the like, and mixtures thereof. Other fatty acids include, but are not limited to, decenoic acid, docosanoic acid, stearic acid, palmitic acid, lauric acid, myristic acid, and the like, and mixtures thereof. In one embodiment the fatty acids employed include, but are not limited to, hydrogenated palm oil, hydrogenated castor oil, stearic acid, hydrogenated cottonseed oil, palmitic acid, and mixtures thereof. Long chain monohydric alcohols include, but are not limited to, cetyl alcohol, stearyl alcohol or mixtures thereof. Waxes are esters of fatty acids with long chain monohydric alcohols. Natural waxes are often mixtures of such esters, and may also contain hydrocarbons. Waxes are low-melting organic mixtures or compounds having a high molecular weight and are solid at room temperature. Waxes may be hydrocarbons or esters of fatty acids and alcohols. Waxes employed in the present invention include, but are not limited to, natural waxes, such as animal waxes, vegetable waxes, and petroleum waxes (i.e., paraffin waxes, microcrystalline waxes, petrolatum waxes, mineral waxes), and synthetic waxes. Specific examples include, but are not limited to, spermaceti wax, carnauba wax, Japan wax, bayberry wax, flax wax, beeswax, Chinese wax, shellac wax, lanolin wax, sugarcane wax, candelilla wax, paraffin wax, microcrystalline wax, petrolatum wax, carbowax, and the like, or mixtures thereof. Mixtures of these waxes with the fatty acids may also be used. Waxes are also monoglyceryl esters, diglyceryl esters, or triglyceryl esters (glycerides) and derivatives thereof formed from a fatty acid having from about 10 to about 22 carbon atoms and glycerol, wherein one or more of the hydroxyl groups of glycerol is substituted by a fatty acid. Glycerides employed in the present invention include, but are not limited to, glyceryl monostearate, glyceryl distearate, glyceryl tristearate, glyceryl dipalmitate, glyceryl tripalmitate, glyceryl monopalmitate, glyceryl dilaurate, glyceryl trilaurate, glyceryl monolaurate, glyceryl didocosanoate, glyceryl tridocosanoate, glyceryl monodocosanoate, glyceryl monocaproate, glyceryl dicaproate, glyceryl tricaproate, glyceryl monomyristate, glyceryl dimyristate, glyceryl trimyristate, glyceryl monodecenoate, glyceryl didecenoate, glyceryl tridecenoate, glyceryl behenate, polyglyceryl diisostearate, lauroyl macrogolglycerides, oleyl macrogolglycerides, stearoyl macrogolglycerides, and the like, or mixtures thereof. Resins employed in the compositions of the present invention include, but are not limited to, shellac and the like or any combinations thereof. In one embodiment the non-polymeric release modifier employed includes, but is not limited to, Cutina® (Hydrogenated castor oil), Hydrobase® (Hydrogenated soybean oil), Castorwax® (Hydrogenated castor oil, Croduret® (Hydrogenated castor oil), Carbowax®, Compritol® (Glyceryl behenate), Sterotex® (Hydrogenated cottonseed oil), Lubritab® (Hydrogenated cottonseed oil), Apifil® (Wax yellow), Akofine® (Hydrogenated cottonseed oil), Softisan® (Hydrogenated palm oil), Hydrocote® (Hydrogenated soybean oil), Corona® (Lanolin), Gelucire® (Macrogolglycerides Lauriques), Precirol® (Glyceryl Palmitostearate), Emulcire™ (Cetyl alcohol), Plurol® diisostearique (Polyglyceryl Diisostearate), Geleol® (Glyceryl Stearate), and mixtures thereof. In a further embodiment the non-polymeric release modifier employed includes, but is not limited to, Compritol®, Sterotex®, Lubritab®, stearic acid, cetyl alcohol, or mixtures thereof.

The controlled release coatings of the present invention may be used in admixture with at least one pharmaceutically acceptable excipient, such as but not limited to, plasticizers, pigments and the like or any mixtures thereof. Suitable plasticizers include, but are not limited to, dibutyl sebacate, propylene glycol, polyethylene glycol, polyvinyl alcohol, triethyl citrate, acetyl triethyl citrate, acetyl tributyl citrate, tributyl citrate, triacetin or the like or any combinations thereof. In an embodiment, the drug-resin complexes are directly coated with the water-insoluble controlled release coatings. A coating procedure known to a person skilled in the art, which provides a substantially complete coating on the drug-resin complexes or impregnated drug-resin complexes without significant agglomeration of the drug-resin complex particles, may be used. Coating to the drug-resin complexes may be applied using wet granulation, dry granulation, melt granulation, melt coating, physical mixing, spray coating and the like. Coatings may be applied in a coating pan or with a fluid-bed coating apparatus. The controlled release coatings may be applied from aqueous suspension or organic solvents. Optionally after coating the coated drug-resin complexes may be cured at a suitable temperature and for a suitable amount of time.

In one embodiment, only coated drug-resin complexes are incorporated in the compositions of the present invention. Optimum coat weight and coat thickness may be determined for each drug-resin complex and generally depends on the drug release characteristics of the resin for that particular active agent. In one embodiment the drug-resin complexes are variably coated at different levels of barrier coating of controlled release coatings and the variably coated drug-resin complexes are present in particular proportions in the sustained release compositions. In one embodiment the compositions of the present invention comprise at least two variably coated populations or portions of coated drug-resin complexes or the coated drug-resin complexes are present in the form of at least two populations of variably coated drug-resin complexes. In one embodiment, coated and uncoated drug-resin complexes are incorporated in the formulations of the present invention. The coated and uncoated drug-resin complexes may be present in a ratio from about 1:99 to about 99:1. In one embodiment, entire amount of the dose of the drug may be incorporated in the composition of the present invention in the form of coated drug-resin complex. In another embodiment, about 1% to about 100% of the dose of the drug may be incorporated in the composition of the present invention in the form of coated drug-resin complex. In another embodiment, about 5% to about 95% of the dose of the drug may be incorporated in the composition of the present invention in the form of coated drug-resin complex. In a further embodiment, about 1% to about 99% of the dose of the drug may be incorporated in the composition of the present invention in the form of uncoated drug-resin complex. In another embodiment, about 5% to about 95% of the dose of the drug may be incorporated in the composition of the present invention in the form of uncoated drug-resin complex. In one embodiment drug resin particles may be coated to a weight gain from about 1% to about 95% of the drug resin complexes. In another embodiment drug resin particles may be coated to a weight gain from about 1% to about 90% of the drug resin complexes. In one embodiment drug resin particles may be coated to a weight gain from about 1% to about 85% of the drug resin complexes. In one embodiment drug resin particles may be coated to a weight gain from about 1% to about 80% of the drug resin complexes. In another embodiment at least two populations of variably coated drug-resin complexes may be present in a ratio from about 1:99 to about 99:1. The coated drug-resin complexes may be present in the modified release units of the present invention in an amount of from about 1% to about 100% by weight.

### Modified Release Units

Modified release units or beads of the present invention comprise coated drug-resin complexes discussed above. In one embodiment the coated drug-resin complexes are modified release units. In another embodiment the modified release units comprise coated drug-resin complexes and at least one pharmaceutically acceptable excipient such as, but not limited to, diluents, stabilizers, controlled release coatings or release modifiers, or the like or any combinations thereof. In one embodiment the modified release units of the present invention comprise variably coated drug-resin complexes in particular proportions as discussed under coated drug-resin complexes above. The compositions of the present invention comprise one or more modified release units. The modified release units employed in the present invention are in forms such as, but not limited to, powder, particles, granules, pellets, beads, minitablets, tablets and the like or any combinations thereof. The modified release units may be present in the compositions in an amount from about 5% to about 95% by weight of the composition. In a further embodiment the coated drug-resin complexes and/or modified release units may be overcoated using conventional polymers including, but not limited to, hydroxypropyl methyl cellulose, hydroxypropyl cellulose, carboxymethyl cellulose, polyvinyl alcohol polymethacrylates and the like or combinations thereof, waxes, and combinations thereof; or using controlled release coatings listed above under controlled release coatings. In one embodiment modified release units in the form of drug-resin complexes are incorporated in the modified release pharmaceutical compositions of the present invention. The modified release units are incorporated in the modified release pharmaceutical compositions of the present invention by any of the methods generally known to a person skilled in the art, especially depending on the form of the modified release units being incorporated and the final form of the pharmaceutical composition. In one embodiment, the active agent for modified release present in the formulation of the present invention is not dispersed in a polymeric or wax matrix. In a further embodiment, the active agent for modified release present in the formulation of the present invention is not dispersed in a polymeric or wax matrix in a non-complexed form.

### Modified Release Formulations

For the purpose of the present invention, the terms “controlled release” or “modified release” or “sustained release” or “prolonged release” are interchangeably used. For the purpose of the present invention the terms “compositions” and “formulations” or “dosage forms” are interchangeably used.

Modified release pharmaceutical formulations of the present invention comprise modified release units and at least one pharmaceutically acceptable excipient. The modified release compositions may be formulated for delivery of active agent by any suitable route including, e.g. orally, topically, intraperitoneally, transdermal, sublingually, intramuscularly, transmucosally, rectally, subcutanoeulsly, transnasally or via inhalation. In one embodiment, the sustained release compositions are for oral delivery. The compositions for oral delivery may be in any form, such as, but not limited to, liquid, solid or semi-solid preparations and the like. Liquid preparations for oral administration may be in any form including, but not limited to, suspensions, syrups or the like. Solid preparations for oral administration may be in any form including, but not limited to, capsules, tablets, caplets, orally disintegrating tablets, dispersible tablets, dry suspension for reconstitution, granules, wafers, bite-dispersion tablets and the like or any combinations thereof. The formulations of the present invention are in the form of taste-masked palatable formulations wherein the bitter taste of memantine is not perceived. In one embodiment the modified release formulation of the present invention is a suspension.

The modified release compositions of the present invention comprise at least one pharmaceutically acceptable excipient, depending on the final dosage form to be prepared, such as, but not limited to, binders, disintegrants, superdisintegrants, diluents, salivating agents, surfactants, flavors, sweeteners, colorants, souring agents, viscolizers, glidants, lubricants, solubilizers, stabilizers, suspending agents, preservatives, cosolvents, anti-caking agents, buffers and the like or any combinations thereof. Suitable disintegrants can be selected from, but not limiting to, crospovidone, calcium silicate and starch. Suitable superdisintegrants include, but are not limited to, natural, modified or pregelatinized starch, crospovidone, croscarmellose sodium, sodium starch glycolate, low-substituted hydroxypropyl cellulose. Examples of suitable binders include, but are not limited to, starch, pregelatinized starch, polyvinyl pyrrolidone, copovidone, cellulose derivatives, such as hydroxypropylmethyl cellulose, hydroxypropyl cellulose and carboxymethyl cellulose and their salts. Examples of suitable diluents include, but are not limited to, starch, microcrystalline cellulose, lactose, xylitol, mannitol, maltose, polyols, fructose, guar gum, sorbitol, magnesium hydroxide, dicalcium phosphate, coprocessed mannitol and calcium silicate and the like or any combinations thereof. Examples of lubricants include, but are not limited to, magnesium stearate, calcium stearate, stearic acid, talc, and sodium stearyl fumarate. Suitable glidants includes but are not limited to, colloidal silica, silica gel, precipitated silica, or combinations thereof. Suitable salivating agents include, but are not limited to, micronised polyethylene glycol, sodium chloride or precipitated micronised silica. Examples of solubilizers include, but are not limited to cetostearyl alcohol, cholesterol, diethanolamine, ethyl oleate, ethylene glycol palmitostearate, glycerin, glyceryl monostearate, isopropyl myristate, lecithin, medium-chain glyceride, monoethanolamine, oleic acid, propylene glycol, polyoxyethylene alkyl ether, polyoxyethylene castor oil glycoside, polyethylene sorbitan fatty acid ester, polyoxyethylene stearate, propylene glycol alginate, sorbitan fatty acid ester, stearic acid, sunflower oil, triethanolmine, or combinations thereof. Souring agents include, but are not limited to, monosodium fumarate and/or citric acid.

The compositions of the present invention may also include stabilizers such as, but not limited to, those described above under drug-resin complexes. Suitable viscolizers include, but are not limited to, coprocessed microcrystalline cellulose such as but not limited to, Avicel RC591, Avicel CL-611, D-sorbitol solution, polyalkylene oxides such as, but not limited to polyethylene oxide; cellulose ethers such as, but not limited to hydroxyethyl cellulose, hydroxypropylcellulose, hydroxypropyl methyl cellulose, methyl cellulose, ethyl cellulose, sodium carboxy methylcellulose, calcium carboxymethyl cellulose, microcrystalline cellulose; gums such as but not limited to gum arabic alginates, agar, sodium alginate guar gum, locust bean, carrageenan, tara, gum arabic, tragacanth, pectin, xanthan, gellan, maltodextrin, galactomannan, pusstulan, laminarin, scleroglucan, gum arabic, inulin, karaya, whelan; polyols such as, but not limited to dipropylene glycol, polypropylene glycol, propylene glycol, polyethylene glycol (PEG), sorbitol and glycerol; carbopol, starch and starch-based polymers such as, but not limited to, pregelatinized starch, acrylic acid and methacrylic acid polymers, and esters thereof, maleic anhydride polymers; polymaleic acid; poly(acrylamides); poly(olefinic alcohol)s; poly(N-vinyl lactams); polyoxyethylated saccharides; polyoxazolines; polyvinylamines; polyvinylacetates; polyimines; povidone, vinylpyrrolidone/vinyl acetate copolymer and polyvinyl acetate, mixture of polyvinyl acetate and polyvinylpyrrolidone, chitin, cyclodextrin, gelatin, chitosan and the like or any mixtures thereof.

Suitable surfactants include, but are not limited to, anionic, nonionic, cationic, and zwitterionic surfactants or a mixture thereof. The non-ionic surfactants employed in the composition may include, but are not limited to, ethoxylated fatty acid ester, ethoxylated fatty acid ethers, ethoxylated sorbitan ethers, ethoxylated alkyl-phenols, glycerol esters, glycerol sugar esters, polyoxyethyleneglycerol monolaurate, polyoxyethyleneglycerol monostearate, polyoxyethylene-20-cetyl stearate, polyoxyethylene-25-cetyl stearate, polyoxyethylene (25)-oxypropylene monostearate, polyoxyethylene-20-sorbitan monopalmitate, poly-oxyethylene-16-tert-octylphenol, polyoxyethylene-20-cetyl ether, polyethylene glycol(1000) monocetyl ether, ethoxylated castor oil, polyoxyethylene sorbitol-lanolin derivatives, polyoxyethylene(25)propylene glycol stearate, polyoxyethylenesorbitol esters, polyoxyethylene-20-sorbitan monopalmitate, polyoxyethylene-16-tert-octylphenol, polyoxyethylene-20-cetyl ether, glycyeryl undecylenate and Polysorbate 60, capmul (medium chain glyceride), peceol (glyceryl monooleate), glyceryl laurate and glyceryl caprylate (Capmul MCM), PEG sorbitan fatty acid esters like PEG-20 sorbitan monolaurate (Tween 20), PEG-20 sorbitan monostearate (Tween 60), PEG-20 sorbitan monooleate (Tween 80), sorbitan fatty acid esters like sorbitan monolaurate (Span 20), glyceryl stearate (Cithrol GMS) or the like and mixtures thereof. Suitable cationic surfactants include, but are not limited to, quaternary ammonium compounds, alkylamidoamines and quaternary ester compounds, distearyl dimethyl ammonium chloride, dimyristyl dimethyl ammonium chloride, dipalmityl dimethyl ammonium chloride or the like and mixtures thereof. Suitable anionic surfactants include, but are not limited to, fatty alcohol sulfates, alpha olefin sulfonates, sulfosuccinates, phosphate esters, carboxylates, sarcosinates, alkyl benzene sulfonates, alkyl sulfonates, olefin sulfonates, alkyl ethersulfonates, glycerol ethersulfonates, a-methyl estersulfonates, sulfonic fatty acids, alkyl sulfates, fatty alcohol ethersulfates, glycerol ethersulfates, mixed hydroxy ethersulfates, monoglyceride (ether)sulfates, fatty acid amide (ether)sulfates, sulfosuccinates, sulfosuccinamates, sulfotriglycerides, amide soaps, ether carboxylic acids, isethionates, sarcosinates, taurides, alkyl oligoglycoside sulfates, alkyl (ether)phosphates or the like and mixtures thereof. Suitable zwitterionic surfactants employed include, but are not limited to, N-alkyl-N,N-dimethyl ammonium glycinates, for example cocoalkyl dimethyl ammonium glycinate, N-acyl aminopropyl-N,N-dimethyl ammonium glycinates, cocoacyl aminoethyl hydroxyethyl carboxymethyl glycinate or the like and mixtures thereof.

Further, the composition of the present invention may further comprise a preservative such as but not limited to methyl parahydroxybenzoate, propyl parahydroxybenzoate and sodium benzoate. Suitable cosolvent that may be used includes, but is not limited to, ethanol and polyhydric alcohols such as, but not limited to, glycerin, propylene glycol, low molecular weight polyethylene glycols, and mixtures thereof. Suitable pH modifiers or buffering agents may be employed such as but not limited to, citric acid, fumaric acid and the like or combinations thereof. Further anti-caking agents that may be optionally incorporated include, but are not limited to, colloidal silicon dioxide, tribasic calcium phosphate, powdered cellulose, magnesium trisilicate, starch, and mixtures thereof. Suitable sweetening agent includes, but is not limited to, aspartame, stevia extract, glycyrrhiza, saccharine, saccharine sodium, acesulfame, sucralose, dipotassium glycyrrhizinate, galactose, fructose, high fructose corn syrup, dextrose, sucrose, sugar, maltose, partially hydrolyzed starch, corn syrup solids, sorbitol, xylitol, mannitol and the like or mixtures thereof. The compositions may comprise one or more natural and/or artificial flavors such as, but not limited to, mint flavour, orange flavour, lemon flavors, strawberry aroma, vanilla flavour, raspberry aroma, cherry flavor, tutty frutty flavor, magnasweet 135, key lime flavor, grape flavor, trusil art 511815, and fruit extracts and the like. Suitable colorants include, but are not limited to, pigments and dyes such as FD&C Red, FD&C Yellow, FD&C Green, and FD&C Blue and the like or combinations thereof. In one of the embodiment, the solid dosage form of the present invention may be optionally coated. Surface coating may be employed for aesthetic purposes or for dimensionally stabilizing the compressed dosage form. The coating may be carried out using any conventional technique employing conventional ingredients suitable for oral use. A surface coating can, for example, be in the form of a film using conventional polymers including, but not limited to, hydroxypropyl methyl cellulose, hydroxypropyl cellulose, carboxymethyl cellulose, polyvinyl alcohol polymethacrylates and the like, and combinations thereof. In another embodiment of the present invention, the composition may be optionally coated with a functional coat. The functional coat may be applied using coating agents including, but not limited to, hydrophilic polymers, hydrophobic polymers, waxes, and the like, or mixtures thereof, either alone or in combination, along with plasticizers, colorants, opacifiers etc. The functional coat may help provide the desired drug release profile. The modified release compositions of the present invention can be readily formulated according to methods well known to those skilled in the art. Method of preparation of the compositions of the present invention depends on the final dosage form desired.

The compositions of the present invention provide modified release of the active agent in-vitro and in-vivo for up to about 24 hours. In one embodiment the compositions of the present invention provide modified release of the active agent in-vitro and in-vivo for up to about 12 hours. In another embodiment the compositions of the present invention provide modified release of the active agent in-vitro and in-vivo for up to about 8 hours. In another embodiment, the compositions of the present invention provide modified release of the active agent in-vitro and in-vivo for about 6 hours. In another embodiment, the compositions of the present invention provide modified release of the active agent in-vitro and in-vivo for about 4 hours to about 24 hours. In one embodiment, the modified release composition of the present invention is for at least once daily administration.

In a further embodiment is provided use of the modified release compositions of the present invention for the prevention, treatment, management or mitigation of various disease conditions or disorders. In one embodiment is provided use of the modified release compositions of the present invention for the treatment, management or alleviation of various progressive neurodegenerative disorders such as dementia of Alzheimer's disease, Parkinson's disease, spasticity, autism, attention deficit/hyperactivity disorder (ADHD) and other autistic spectrum disorders. NMDA receptor antagonist are effective in the treatment of Huntington's disease, amyotrophic lateral sclerosis (ALS), AIDS-related dementia, schizophrenia, motor neurons diseases and CNS and brain injuries resulting from a number of causes including stroke, trauma and neurosurgery. In another embodiment is provided use of the modified release compositions of the present invention for the manufacture of a medicament for the prevention, treatment, management or alleviation of various progressive neurodegenerative disorders such as dementia of Alzheimer's disease, Parkinson's disease, spasticity, autism, attention deficit/hyperactivity disorder (ADHD) and other autistic spectrum disorders. NMDA receptor antagonist are effective in the treatment of Huntington's disease, amyotrophic lateral sclerosis (ALS), AIDS-related dementia, schizophrenia, motor neurons diseases and CNS and brain injuries resulting from a number of causes including stroke, trauma and neurosurgery. In a still further embodiment is provided use of the modified release compositions of the present invention for the manufacture of a medicament for the treatment, management or alleviation of various progressive neurodegenerative disorders such as dementia of Alzheimer's disease, Parkinson's disease, spasticity, autism, attention deficit/hyperactivity disorder (ADHD) and other autistic spectrum disorders. NMDA receptor antagonist are effective in the treatment of Huntington's disease, amyotrophic lateral sclerosis (ALS), AIDS-related dementia, schizophrenia, motor neurons diseases and CNS and brain injuries resulting from a number of causes including stroke, trauma and neurosurgery and general malaise associated therewith is provided which comprises administering to the subject in need thereof modified release composition of the present invention.

In another embodiment, the formulation of present invention comprises one or more active agents. In one embodiment, the second active agent in addition to the Anti-Alzheimer's agent or NMDA receptor antagonist may be present in the dosage form in immediate release or modified release form. In a further embodiment, the second active agent in addition to the Anti-Alzheimer's agent may be present in the dosage form in the form of a complex with ion exchange resin. In another embodiment, the second active agent in addition to the Anti-Alzheimer's agent may not be present in the dosage form in the form of a complex with ion exchange resin. In one embodiment, the second active agent in addition to the Anti-Alzheimer's agent may be present in the dosage form in the form of a coated drug-resin complex. In another embodiment, the second active agent in addition to the Anti-Alzheimer's agent may not be present in the dosage form in the form of a coated drug-resin complex. In one embodiment, the additional active agent may be present in the modified release units. In another embodiment, the additional active agent may not be present in the modified release units. In another embodiment, the formulations of the present invention comprise one or more Anti-Alzheimer's agents. In one embodiment, the formulations of the present invention comprising one or more Anti-Alzheimer's agents may comprise at least one Anti-Alzheimer's agent in modified release form while the other may be for immediate release. In one embodiment, the formulations of the present invention comprising one or more Anti-Alzheimer's agents, at least one Anti-Alzheimer's agent may be delivered in a modified release form, and at least one may be delivered in immediate release form. In another embodiment, the second active agent is different than the first active agent that is delivered in a modified manner. Such a second active agent includes, but is not limited to, the list of active agents discussed above under active agents. In one embodiment, the formulation of present invention comprises a combination of NMDA receptor antagonist and acetylcholinesterase inhibitor. In a further embodiment, the formulation of present invention comprises a combination of NMDA receptor antagonist and acetylcholinesterase inhibitor wherein NMDA receptor antagonist is in modified release form, while the acetylcholinesterase inhibitor is in immediate release form. In a further embodiment, memantine hydrochloride is in modified release form, while donepezil hydrochloride is delivered in immediate release form.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed, but that the invention will include all embodiments falling within the scope thereof. Details of the present invention, including its objects and advantages, are provided in the non-limiting exemplary illustrations below.

### EXAMPLES

### Example 1

### Memantine Modified Release Formulation

TABLE 1
Composition of Memantine modified release formulation

| Ingredients | mg/5 ml |
|---|---|
| Memantine Hydrochloride | 28 |
| Sodium polystyrene sulfonate, USP | 56 |
| Polyethylene glycol, USPNF | 34 |
| Sodium metabisulphite, USPNF | 22 |
| Ethyl cellulose, USP | 21.81 |
| Glycerin, USPNF | 50 |
| Triacetin, USPNF | 6.51 |
| Pharma grade sugar | 1750 |
| Methylparaben, USPNF | 5 |
| Propylparaben, USPNF | 0.5 |
| High fructose corn syrup, USPNF | 850 |
| Xanthan gum, USPNF | 11 |
| Orange flavor | 9 |
| FD & C Yellow | 0.375 |
| FD & C Red | 0.375 |
| Polyoxyethylene sorbitan fatty acid | 10 |
| esters, USP | |
| Citric acid anhydrous, USP | 7.5 |
| Purified water | q.s. |

Procedure:

(i) Memantine Hydrochloride was complexed with sodium polystyrene sulfonate in water under stirring. The drug-resin complex formed was filtered, dried and solvated with polyethylene glycol. This treated drug-resin complex was then coated with ethyl cellulose to give modified release units. These units were dispersed in glycerine. (ii) Suspension base was prepared by dissolving methylparaben, propylparaben, sodium metabisulphite, triacetin in purified water at 85-90° C. Pharma grade sugar was added to above solution under stirring and solution was subsequently cooled to room temperature. Xanthan gum was dispersed in high fructose corn syrup under stirring. This dispersion was added to the above sugar syrup under stirring to get uniform dispersion. Solution of color and flavor was then added. (iii) The modified release units of step i) were then added to the suspension base along with polyoxyethylene sorbitan fatty acid esters and citric acid. Final volume was adjusted with purified water.

This memantine suspension is palatable and has a desired modified release profile.

### Example 2

### Memantine Modified Release Formulation

TABLE 2
Composition of Memantine modified release formulation

| Ingredients | mg/5 ml |
|---|---|
| Memantine Hydrochloride | 21 |
| Donepezil Hydrochloride | 5 |
| Sodium polystyrene sulfonate, USP | 52 |
| Polyethylene glycol, USPNF | 38 |
| Tocopherol, USPNF | 10 |
| Ethyl cellulose, USPNF | 20 |
| Glycerin, USPNF | 50 |
| Pharma grade sugar, USPNF | 1780 |
| Methylparaben, USPNF | 5 |
| Propyl paraben, USPNF | 0.5 |
| High fructose corn syrup, USPNF | 850 |
| Xanthan gum, USPNF | 11 |
| Orange flavor | 9 |
| FD & C Yellow | 0.375 |
| FD & C Red | 0.375 |
| Sorbitan monolaurate, USP | 10 |
| Citric acid anhydrous, USP | 7.5 |
| Purified water | q.s. |

Procedure:

(i) Memantine Hydrochloride was complexed with sodium polystyrene sulfonate in water under stirring. The drug-resin complex formed was filtered, dried and solvated with polyethylene glycol. This treated drug-resin complex was then coated with ethyl cellulose to give modified release units. These units were dispersed in glycerine. Donepezil Hydrochloride was also complexed with sodium polystyrene sulfonate in water under stirring. The drug-resin complex formed was filtered, dried and solvated with polyethylene glycol. (ii) Suspension base was prepared by dissolving methylparaben, propylparaben, tocopherol in purified water at 85-90° C. Pharma grade sugar was added to above solution under stirring and solution was subsequently cooled to room temperature. Xanthan gum was dispersed in high fructose corn syrup under stirring. This dispersion was added to the above sugar syrup under stirring to get uniform dispersion. Solution of color and flavor was then added. (iii) The modified release units of memantine and the ion exchange resin complexes of donepezil prepared in step i) were then added to the suspension base along with sorbitan monolaurate and citric acid. Final volume was adjusted with purified water.

We claim:

1) A modified release composition comprising plurality of modified release units comprising at least one anti-Alzheimer's agent, at least ion exchange resin and at least one release modifier.

2) The composition of claim 1, wherein the anti-Alzheimer's agent is NMDA receptor antagonist, acetyl cholinesterase inhibitor, acetylcholine synthesis modulator, acetylcholine storage modulator, acetylcholine release modulator, Aβ inhibitor, Aβ plaque removal agent, inhibitor of Aβ plaque formation, inhibitor of amyloid precursor protein processing enzyme, β-amyloid converting enzyme inhibitor, β-secretase inhibitor, γ-secretase modulator, nerve growth factor agonist, hormone receptor blockade agent, neurotransmission modulator, or any combination thereof.

3) The composition of claim 2, wherein the anti-Alzheimer's agent is in the form of free base, free acid, pharmaceutically acceptable salt, ester, prodrug, metabolite, active metabolite, derivative, polymorph, solvate, hydrate, enantiomer, optical isomer, tautomer or racemic mixture thereof, or any combination thereof.

4) The composition of claim 3, wherein the anti-Alzheimer's agent is memantine, in the form of free base, pharmaceutically acceptable salt, ester, prodrug, metabolite, active metabolite, derivative, polymorph, solvate, hydrate, enantiomer, optical isomer, tautomer or racemic mixture thereof, or any combination thereof.

5) The composition of claim 4, wherein the anti-Alzheimer's agent is memantine hydrochloride.

6) The composition of claim 1 wherein the ion exchange resin is a cation exchange resin or an anion exchange resin or any combination thereof.

7) The composition of claim 6, wherein the cation exchange resin is a copolymer of methacrylic acid and divinylbenzene, sodium polystyrene sulfonate resin, sulfonated copolymer of styrene and divinylbenzene, crosslinked polyacrylic acid resin, polyacrylate resin, crosslinked carboxylic acid resin, crosslinked sulfonic acid resin, crosslinked phosphonic acid resin, zeolite or any combination thereof.

8) The composition of claim 1, wherein anti-Alzheimer's agent is present in the form of complex with the ion exchange resin.

9) The composition of claim 1, wherein the release modifier is at least one polymeric release modifier or at least one non-polymeric release modifier or any combination thereof.

10) The composition of claim 9, wherein the polymeric release modifier is a cellulose derivative, a saccharide or polysaccharide, a poly(oxyethylene)-poly(oxypropylene) block copolymer, a vinyl derivative or polymer or copolymer thereof, a polyalkylene oxide and derivative thereof, a maleic copolymer, a acrylic acid derivative or any combination thereof.

11) The composition of claim 10, wherein the polymeric release modifier is ethyl cellulose, methylcellulose, hydroxypropylmethylcellulose, hydroxypropyl cellulose, hydroxyethyl cellulose, hydroxymethyl cellulose, hydroxypropyl ethylcellulose, carboxymethylethyl cellulose, carboxy ethylcellulose, carboxymethyl hydroxyethylcellulose, hydroxyethylmethyl carboxymethyl cellulose, hydroxyethyl methyl cellulose, carboxymethyl cellulose, methylhydroxyethyl cellulose, methylhydroxypropyl cellulose, carboxymethyl sulfoethyl cellulose, sodium carboxymethyl cellulose, cellulose acetate, cellulose acetate phthalate, cellulose acetate butyrate, hydroxypropylmethylcellulose acetate succinate, hydroxypropylmethylcellulose phthalate, hydroxymethyl ethylcellulose phthalate, cellulose acetate phthalate, cellulose acetate succinate, cellulose acetate maleate, cellulose acetate trimelliate, cellulose benzoate phthalate, cellulose propionate phthalate, methylcellulose phthalate, ethylhydroxy ethylcellulose phthalate, guar gum, xanthan gum, gum arabic, tragacanth, polyvinylacetate aqueous dispersion, copolymers of vinyl pyrrolidone, copolymers of polyvinyl alcohol, mixture of polyvinyl acetate and polyvinylpyrrolidone, polyvinyl alcohol phthalate, polyvinylacetal phthalate, polyvinyl butylate phthalate, polyvinylacetoacetal phthalate, polyvinylpyrrolidone, polyethylene oxide methacrylic acids, polymethacrylic acids, polyacrylates, polymethacrylates, vinylacetate.maleic acid anhydride copolymer, styrene.maleic acid anhydride copolymer, styrene.maleic acid monoester copolymer, vinylmethylether.maleic acid anhydride copolymer, ethylene.maleic acid anhydride copolymer, vinylbutylether.maleic acid anhydride copolymer, acrylonitrile.methyl acrylate.maleic acid anhydride copolymer, butyl acrylate.styrene.maleic acid anhydride copolymer or any combinations thereof.

12) The composition of claim 1, wherein the release modifier is present in the form of release modifying coating.

13) The composition of claim 1, wherein the modified release units further comprise at least one pharmaceutically acceptable excipient, said pharmaceutically acceptable excipient being a diluent, a stabilizer, or a release modifier or any mixtures thereof.

14) The composition of claim 1 wherein the modified release composition further comprises at least one pharmaceutically acceptable excipient; said pharmaceutically acceptable excipient being binder, disintegrant, superdisintegrant, diluent, salivating agent, surfactant, flavor, sweetener, colorant, souring agent, viscolizer, glidant, lubricant, solubilizer, stabilizer, suspending agent, preservative, cosolvent, anti-caking agent, buffer or any combination thereof.

15) The composition of claim 1 wherein the modified release composition is in the form of a liquid, a solid or a semisolid preparation; said liquid preparation being suspension and said solid preparation being capsule, tablet, caplet, orally disintegrating tablet, dry suspension for reconstitution, granule, wafer or bite-dispersion tablet.

16) The composition of claim 1 wherein the modified release units comprise:
a) anti-Alzheimer's agent and ion exchange resin complex; and
b) release modifying coating.

17) The composition of claim 1, wherein the modified release composition further comprises an additional active agent.

18) The composition of claim 17, wherein the additional active agent is delivered in an immediate or sustained release manner.
